(19) United States
(12) Patent Application Publication
(10) Pub. No.: US 2024/0081142 A1
(43) Pub. Date: Mar. 7, 2024

(54) LIGHTING DEVICE

(71) Applicant: Matrix Sensors, Inc., San Diego, CA (US)

(72) Inventor: David K. Britt, El Cerrito, CA (US)

(73) Assignee: Matrix Sensors, Inc., San Diego, CA (US)

(21) Appl. No.: 18/376,441

(22) Filed: Oct. 4, 2023

Related U.S. Application Data
(63) Continuation of application No. 16/798,075, filed on Feb. 21, 2020.
(60) Provisional application No. 62/809,333, filed on Feb. 22, 2019.

(51) Int. Cl. H10K 85/30; C07D 487/22; C09K 11/06

(52) U.S. Cl. CPC H10K 85/371; C07D 487/22; C09K 11/06; C09K 2211/1029; C09K 2211/188; H10K 50/11

(57) ABSTRACT

A lighting device comprises an emission layer, and first and second electrodes between which a voltage can be applied to generate an electric field in at least part of the emission layer. The emission layer comprises a luminescent film composed of at least one crystal comprising repeating structural units of a crystalline framework material. The crystal has at least one crystallographic axis that is aligned substantially parallel to a planar surface of the luminescent film with a maximum deviation of up to +/−35° from this parallel orientation. Luminescent emitters are arranged in at least 30% of the repeating structural units of the crystalline framework material such that the transition dipole moments of the luminescent emitters are configured in a substantially parallel orientation relative to the crystallographic axis with a maximum deviation of up to +/−350 from this parallel orientation.

### FIELD OF THE INVENTION

The present disclosure relates generally to a lighting device, and in particular to a lighting device having improved orientation of the emitted light.

This patent application is a continuation of U.S. patent application Ser. No. 16/798,075 filed Feb. 21, 2020 which application claims the benefit of U.S. provisional patent application 62/809,333 filed on Feb. 22, 2019. All of these applications are hereby incorporated by reference in their entirety.

### INTRODUCTION

Semiconductor layers employed in organic semiconductor components are primarily amorphous. The lack of order in these amorphous layers is a disadvantage for a variety of physical properties, for example for the important conductivity of the semiconductor layers. A very specific disadvantage for the efficiency of the components arises, however, in the area of the light-emitting components, more particularly of organic light-emitting diodes. In these components, the disoriented emission of light harbors a large loss factor for the external quantum efficiency, i.e., the fraction of photons generated that is also actually emitted to the outside. Existing OLEDs feature an external quantum efficiency, without outcoupling aids, of not more than about 20%.

The efficiency of organic light-emitting diodes (OLEDs) is measured using the light yield. Besides the internal quantum efficiency, which is determined by parameters inherent in the material of the emitters and by the self-absorption properties of the semiconductor layers, optical parameters make a large contribution to a reduction in the external quantum efficiency—that is, the photons actually emitted to the outside. These parameters are, for example, incoupling losses into the glass substrate, the excitation of waveguide modes, and the losses due to excitation of plasmons in the reflecting electrodes. In order to minimize the losses due to undirected emission within the OLED, reflective electrodes have been fabricated from reflective material such as aluminum or silver, for example, leading to high reflection of the photons generated. This solution, however, is not very effective, since the excitation of plasmons in the electrodes means that there are again large losses of the generated light quanta. These losses due to plasmons amount to approximately 30%. These losses can only be reduced if a lower proportion of the photons generated actually impinges on the reflective electrodes. The emission would have to be directed in such a way that the number of emitting dipole vectors normal to the reflective electrodes becomes minimal.

A fundamental barrier to the orientation of the emitters, however, is that it is necessary to know the direction in which a molecule is emitting, in relation to its internal molecular coordinate system. Depending on the spatial orientation of the HOMO (highest occupied molecular orbital) and LUMO (lowest unoccupied molecular orbital), the first excited state has a different dipole moment from the ground state. The emission dipole correlates with the dipole moment in the ground state.

Traditional OLED device configurations are close to theoretical efficiency (e.g., >97%). It is known that perfect alignment of the phosphor molecules within OLED devices would increase the theoretical maximum efficiency by 50%, which opens up massive potential for improvement over existing devices. This efficiency increase arises from the fact that photons emitted from a phosphor molecule are perpendicular to the dipole of the transition that leads to emission. Photons emitted parallel to the plane of the device are lost to surface plasmon and other lossy modes.

### SUMMARY

According to an aspect, a lighting device comprises an emission layer, and first and second electrodes between which a voltage can be applied to generate an electric field in at least part of the emission layer. The emission layer comprises a luminescent film composed of at least one crystal comprising repeating structural units of a crystalline framework material. The crystal has at least one crystallographic axis that is aligned substantially parallel to a planar surface of the luminescent film with a maximum deviation of up to +/−35° from this parallel orientation. Luminescent emitters are arranged in at least 30% of the repeating structural units of the crystalline framework material such that the transition dipole moments of the luminescent emitters are configured in a substantially parallel orientation relative to the crystallographic axis with a maximum deviation of up to +/−350 from this parallel orientation.

### BRIEF DESCRIPTION OF THE DRAWINGS

FIG. 1 is a schematic diagram of Tetraphenylporphyrin platinum(II) (TTP-Pt), a known luminophore. The transition dipole of TTP-Pt lies in the plane of the molecule.

FIG. 2 is a schematic block diagram of an OLED device having an emission layer comprising a MOF film with tetraphenylporphyrin platinum(II) (TPP-Pt). The transition dipole of TPP-Pt lies at a predetermined angle parallel to the emission layer and device surface.

FIG. 3 is a schematic block diagram of an emission layer comprising MOF crystals with a sheet-like geometry whose orientation is substantially parallel to the surface of a substrate.

FIG. 4 shows the crystal structure of a Cu₂(TCPP) MOF. The TCPP unit, in which a platinum ion is bound at the center of the porphyrin moiety, is complexed to eight copper ions, which form (Cu₂O₈C₄) clusters referred to as “paddlewheel” structural units.

FIG. 5 is a schematic block diagram of an OLED device with an emission layer formed by an oriented MOF film with oriented phosphors.

FIG. 6 shows diagrams of exemplary luminescent emitters (e.g., phosphors) with their transition dipole moments indicated by arrows.

### DETAILED DESCRIPTION

Aspects of the present disclosure include a luminescent film that includes a crystalline framework material comprising repeating structural units and a plurality of luminescent emitters each configured in a repeating structural unit of the crystalline framework material to have a non-random orientation relative to the crystalline framework material. Aspects of the present disclosure further include a light-emitting device and the luminescent film suitable for use in an emission layer of the device. The luminescent moieties or molecules of the present disclosure are maintained in a desired orientation by incorporating them into a crystalline framework material. The combination of the light-emitting device and luminescent film of the present disclosure may be suitable in an apparatus for illumination and/or display. The combination of the light-emitting device and luminescent film of the present disclosure increases energy efficiency of the emission of light from the light-emitting device.

According to a first aspect, the present disclosure provides a method for producing a film comprising at least one emitter material, and in some cases, a luminescent emitter such as a luminescent moiety or a luminophore molecule. In some cases, emitter materials include luminescent moieties or luminophores (e.g., a fluorophore or phosphor). The orientation of the luminophore is controlled such that the transition dipole moment of that luminophore is oriented in a predetermined orientation, e.g. parallel to a planar surface of the film or parallel to the planar surface of a substrate on which the film is deposited or grown. Transition dipole moments are also designated hereinafter as transition dipoles or dipole moments for short.

In some embodiments, a luminescent film comprises a crystalline framework material. In some cases, the crystalline framework material comprises repeating structural units having a uniform orientation relative to a planar surface of the film. In some cases, the luminescent film comprises a plurality of luminescent emitters. In some cases, each of the plurality of luminescent emitters are configured in a repeating structural unit of the crystalline framework material to have a non-random orientation relative to the crystalline framework material.

In some embodiments, the film is composed of a plurality of crystals composed of the crystalline framework material and each having at least one crystallographic axis aligned substantially parallel to the planar surface of the film, wherein a plurality of the repeating structural units of each crystal comprise a luminescent emitter having a transition dipole moment configured in a substantially parallel orientation relative to the at least one crystallographic axis.

In some embodiments, the repeating structural units of the crystalline framework material each comprise a structurally integrated luminescent emitter (e.g., a luminescent emitter that is part of, and essential to, the structure of the crystalline framework).

In some embodiments, the repeating structural units can be referred to as unit cells of the crystalline framework material.

In some embodiments, a crystalline framework material (e.g., a MOF, COF, or porous coordination polymer) incorporates the luminophore such that: (a) the unit cell of the crystalline framework material has a known orientation with respect to a substrate when grown or cast as a film on the substrate; and (b) the luminophore has a known orientation with respect to the unit cell of the crystalline framework material. The smallest group of particles in the crystalline framework material that constitutes the pattern repeated by translations is the unit cell of the structure. In some cases, the combination of conditions (a) and (b) results in the transition dipole moment of the luminophore having a predetermined orientation (e.g., parallel) with respect to a planar surface of the film and the surface of the substrate on which the film is grown or cast.

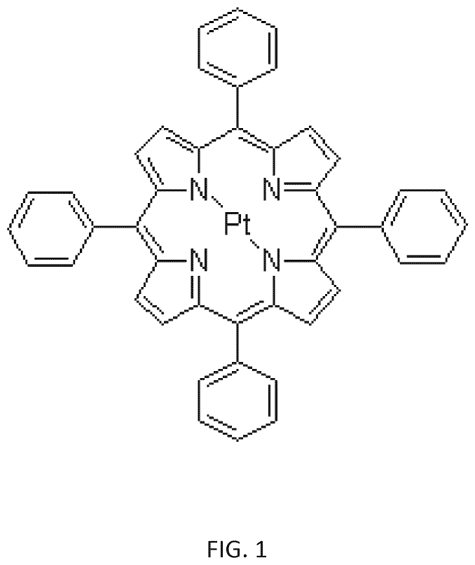

FIG. 1 shows a phosphor tetraphenylporphyrin platinum (II) (TTP-Pt). The transition dipole moment of TTP-Pt lies in the plane of the molecule. Having identified a luminescent molecule with a given transition dipole moment, next a MOF structure is identified that is suitable to align or orient the transition dipole of that luminescent molecule in a desired orientation with respect to a surface of the film and/or the surface of the substrate of a light-emitting device on which the film is deposited or grown.

FIG. 3 shows a schematic block diagram of an oriented MOF film 20 grown or deposited on the planar surface of a substrate 18. The film 20 is a layer comprising MOF crystals or crystallites 22 having a sheet-like geometry whose orientation is substantially parallel to the surface of the substrate 18. Metal-organic frameworks (MOFs), known variously as porous coordination frameworks and porous coordination polymers, make up a class of crystalline, porous materials that may be formed or deposited on the substrate 18 as a polycrystalline thin film 20. The formation or deposition is performed such that the film 20 has, in some cases, a crystallographic orientation with respect to the substrate 18. A film so formed or deposited is referred to as an “oriented film”. As a result of that particular crystallographic orientation, the various components of the MOF material in the oriented film 20 also have a particular orientation with respect to the substrate 18.

The MOF or COF material comprises at least one luminescent emitter, such as a luminescent moiety or a luminophore (e.g., phosphor molecules or fluorophore molecules). An oriented film of such a MOF imparts a particular orientation on all of its components (e.g., unit cells with luminescent emitters). Thus, a particular MOF structure may be designed such that an oriented film of that MOF imparts a desired orientation on the luminescent emitters (e.g., such that the transition dipoles of the luminescent emitters have a substantially parallel orientation to the planar surface of the film) that are held in the correct position by the unit cells to achieve that orientation. The oriented film typically has a thickness in the range of 1 nm to 1 um, and in some cases, in the range of 1 nm to 1,000 nm, 1 nm to 500 nm, or in some cases in the range of 2 nm up to about 150 nm. The horizontal dimensions are likely dependent upon the particular lighting application (e.g., sized in terms of single pixels in a display, or other sizes).

In some embodiments, the luminescent film of the present disclosure includes an emission layer composed 1) solely of an oriented MOF film with oriented phosphors incorporated into the MOF structure itself or 2) of a MOF film with oriented phosphors within the MOF pores but not incorporated into the MOF structure itself. Embodiments also include emission layers made up of an oriented MOF film of either configuration above, as well as other conductive molecules or polymers, which may be within the pores of the MOF or outside of the MOF structure. Embodiments also include emission layers in which the MOF plays the role of conducting electrons, holes, or both.

In some embodiments, the luminescent film of the present disclosure has a thickness of from 1 nm to 10,000 nm. In some embodiments, the luminescent film has a thickness of from 1 nm to 100 nm, of from 100 nm to 500 nm, of from 500 nm to 1000 nm, of from 1000 nm to 1500 nm, of from 1500 nm to 2000 nm, of from 2000 nm to 2500 nm, of from 2500 to 3000 nm, of from 3000 nm to 3500 nm, of from 3500 nm to 4000 nm, of from 4000 nm to 4500 nm, of from 4500 nm to 5000 nm, of from 5000 nm to 5500 nm, of from 5500 nm to 6000 nm, of from 6000 nm to 6500 nm, of from 6500 nm to 7000 nm, of from 7000 nm to 7500 nm, of from 7500 nm to 8000 nm, of from 8000 nm to 8500 nm, of from 8500 nm to 9000 nm, of from 9000 nm to 9500 nm, of from 9500 nm to 10,000 nm.

### Crystalline Framework Material

Aspects of the present disclosure include a crystalline framework material comprising repeating structural units having a uniform orientation relative to a planar surface of a luminescent film. The present disclosure provides rigid, ordered crystalline framework materials based on repeating structural units having a repeated and uniform orientation. The crystalline framework material can be monocrystalline or polycrystalline. The regularity of the crystalline framework material is useful for achieving control over the orientation of multiple luminescent emitters that are incorporated into the framework (e.g., as described herein). The luminescent emitters which are incorporated into the film and uniformly oriented with respect to the repeating structural units of the crystalline framework material and thus with respect to the larger luminescent film structure, can provide a desirable intensity and direction of light emission from the film.

In some embodiments, the crystalline framework material is a metal-organic framework comprising metallic structural units linked by organic structural units.

In the context of the present disclosure, and in its conventional sense, the term “metal-organic framework” refers to a one-, two-, or three-dimensional coordination polymer including metallic structural units (sometimes also referred to as “inorganic connectors”) and organic structural units (sometimes also referred to as “linkers”), wherein at least one of the metallic structural units is chemically bonded to at least one bi-, tri-or poly-dentate organic structural unit. It is noted that a “metallic structural unit” may refer to any structural unit comprising a metal or a metal oxide, and that an “organic structural unit” may also refer to a modified unit that contains an inorganic moiety coupled to an organic moiety.

In some embodiments, the metal-organic framework comprised in the film or light-emission layer may comprise a single type of metallic structural unit, or different types of metallic structural units. In some embodiments, the metal-organic framework comprised in the electroluminescent compound of the disclosure may comprise a variety of metallic structural units. For example, at least part of the metallic structural units may comprise one or more elements selected from the group consisting of Al, Zn, Cu, Cr, In, Ga, Fe, Sc, Ti, V, Co, Ni, La, Ce, Pr, Nb, Pm, Sm, Eu, Gd, Tb, Dy, Ho, Er, Tm, Yb, Lu, Ac, Th, Pa, U, Np, Pu, Am, Cm, Bk, Cf, Es, Fm, Md, No and Lr, such as one or more elements selected from the group consisting of Al, Zn, Ga, In, Fe, Cr and Sc, or one or more elements selected from the group consisting of Al, In, and Ga. In some embodiments, when at least part of the metallic structural units comprise a rare earth element it is possible to tune the emission wavelength of the electroluminescent compound. In some cases, the metallic structural units of the metal organic framework are metal ions or metal ion clusters of a metal selected from copper, zinc, aluminium, nickel, gadolinium, and the like.

In some embodiments, the metal-organic framework comprised in the film or light-emission layer may comprise a variety of organic structural units. For example, at least part of the organic structural units may comprise a moiety chosen from the group consisting of porphyrins, perylenes, and carboxylates. Non-limiting examples of suitable carboxylates are nitrogen-containing carboxylates such as pyridine-like moieties having one nitrogen atom (pyridines), two nitrogen atoms (imidazoles, bipyridines), three nitrogen atoms (triazoles) or more nitrogen atoms. Nitrogen-containing carboxylates may be used in combination with dicarboxylates and/or tricarboxylates. Further non-limiting examples of suitable carboxylates are oxalic acid, malonic acid, succinic acid, glutaric acid, phthalic acid, isophthalic acid, terephthalic acid, citric acid, trimesic acid, and mixtures thereof.

In some embodiments, the metal-organic framework comprises a repeating structural unit of the formula: [Mₚ(L)ₙ(Y)ₘ] where L is an organic linking unit that connects two or more metal units, and Y is an optional, and the values of p, n and m are readily determined according to the valency of the selected metal (M), and L and/or Y groups. In some embodiments, M is a metal ion and p is 1 to 4; L is a multivalent organic structural unit (e.g., divalent, trivalent, tetravalent); and Y is an optional second ligand (e.g., a pillar ligand, O, OH, H₂O, halogen, acac, solvent, water, dimethyl-formamide (DMF), N-methyl-2-pyrrolidone (NMP), diethyl formamide (DEF), alcohol, amine, thiol, etc.); n is 1 to 4; and m is 0 to 3 (e.g., 0, 0.5, 1).

In some embodiments, the organic structural units of the metal organic framework comprise a substituted aryl or substituted heteroaryl group, e.g., a monocyclic or multicyclic ring system substituted with one or more metal binding groups via an optional linker. In some embodiments, the organic structural units of the metal organic framework comprise a group selected from BDC or TPA (terephthalic acid), BTC (trimesic acid), BTB (4,4′,4″-benzene-1,3,5-triyl-tris(benzoic acid)), and TCPP (4,4,4,4-(porphine-5,10,15,20-tetrayl) tetrakis (benzoic acid)), 2,5-dihydroxyoxidoterephthalic acid, 3,3′-dihydroxybiphenyl-4,4′-carboxylic acid, imidazole, 1,2,3-triazole, 1,2,4-triazole, pyrazine, triazine, dabco, 4,4′-bipyridine, 1,2,4,5-tetrakis(benzene-3,5-dicarboxylato)benzene, 1,3,5-tris(pyrazolato)benzene, 1,3,5-tris(triazolato)benzene, 1,3,5-tris(tetrazolato)benzene, and substituted versions thereof. In some embodiments, said substituted versions are selected from groups extended by addition of additional phenyl rings, and groups where oxygen atoms are substituted for sulfur atoms.

In some embodiments, the repeating structural unit comprises [M₂(L)]. In some embodiments, M is copper or zinc; and L is a tetravalent organic structural unit comprising four metal-binding functional groups (e.g., carboxy groups).

In some embodiments, the repeating structural unit comprises Cu₂(TCPP) where TCPP is 5,10,15,20-tetrakis(4-carboxyphenyl)porphyrin. Other non-limiting examples of the metal-organic framework comprised in the electroluminescent compound of the disclosure are those that are based on aluminium or aluminium carboxylates as metallic structural units, such as MIL-53, MIL-69, MIL-88, MIL-96, MIL-100, MIL-101, and MIL-110, wherein the acronym “MIL” refers to “Materiaux de VInstitut Lavoisier”. Other non-limiting examples of the metal-organic framework comprised in the film or light-emission layer include MIL-53, MIL-69, MIL-88, and MIL-101. In some embodiments, MIL-53, MIL-69, MIL-88, MIL-96, MIL-100, MIL-101, and MIL-110 can be made with different metals, such as, but are not limited to, copper, zinc, aluminum, nickel, gadolinium, Magnesium, cadmium, iron, manganese, cobalt, vanadium, zirconium, titanium, and indium. In some embodiments, the metal-organic framework comprises a unit described by the formula [Cu₃(BTC)₂], [Cu₃(BTC)₂(H₂O)₃], [Zn₄O(BDC)₃], [Zn₄O(BTB)₂], MIL-53, MIL-69, MIL-88, MIL-100, MIL-101, MIL-111, UiO-66, UiO-67, or UiO-68. In some embodiments, the metal-organic framework MIL-53, MIL-69, MIL-88, MIL-100, MIL-101, or MIL-111 includes a metal selected from copper, zinc, aluminum, nickel, gadolinium, magnesium, cadmium, iron, manganese, cobalt, vanadium, zirconium, titanium, and indium. In some embodiments, the metal-organic framework comprises M2(dobpdc), wherein M is Mg, Cu, Zn, Co, Mn, Fe, or Ni. In some embodiments, the metal-organic framework comprises: M-BTT, wherein M is Mg, Cu, Zn, Co, Mn, Fe, Cd, or Ni.

In some embodiments, the crystalline framework material is porous, and each luminescent emitter is non-covalently bound in a pore of the crystalline framework material. In some embodiments, the crystalline framework material is a metal-organic framework, a covalent organic framework, or a porous coordination framework.

In some embodiments, the metal-organic framework may have a one-dimensional porous structure (at least prior to functionalization). The metal organic framework host can give rise to second-harmonic generation (also known as frequency doubling). In accordance with this phenomenon, a material is capable of generating photons with twice the frequency (half the wavelength) of incident photons. In some embodiments, the metal-organic framework may also have a two-dimensional porous structure or a three-dimensional porous structure (at least prior to functionalization). In some embodiments of the crystalline framework material, the luminescent moiety may be grafted to a coordinatively unsaturated metal of the metal-organic framework.

When the metal-organic framework comprises metallic structural units with one or more coordinatively unsaturated metal sites, these sites can be occupied by a luminescent moiety.

In some embodiments, the crystalline framework material forming the film can be used as basis for an emission layer in a light-emitting device. For example, the crystalline framework material, in some embodiments, may be blended with a matrix material. In some cases, the matrix material is at least partially transparent material. Depending on the intended application, the matrix material may comprise one or more compounds selected from the group consisting of polymers (such as electrically conductive polymers or electrically non-conductive polymers), amalgamate pastes, and liquid electrolytes.

In some embodiments, the luminescent film of the present disclosure further comprises a matrix material and one or more optional additives.

In some embodiments, the luminescent film of the present disclosure comprises one or more additives selected from dopants, charge transport compounds, electrolytes, and dyes.

In some embodiments, the matrix material comprises an electrically conductive polymer. The matrix material may comprise one or more electrically conductive polymers. Many different electrically conductive polymers are known, for example polythiophenes, polyanilines, polycarbazoles, polypyrroles, and substituted derivatives thereof. As specific examples can be mentioned poly(3-butylthiophene-2,5-diyl) optionally with a phosphor-based dopant, poly(thiophene-2,5-diyl) which may optionally be bromine terminated, poly(3,4-ethylenedioxythiophene)-poly(styrenesulphonate), poly(p-phenylene vinylene), polyaniline doped with BF₃, polyphenylene sulphide, conductive nylon, polyester urethane, and polyether urethane.

In some embodiments, the matrix material comprises a non-conductive polymer. The matrix material may also comprise one or more non-conductive polymers. Some examples thereof include epoxy resin without hardener and Nafion. Non-conductive polymers can be used in small-scale electroluminescent devices. For example, a small-scale device can be designed using point electrodes, which each are in direct contact with luminescent metal-organic framework crystals. In some embodiments, the matrix material may comprise a polymer that is doped with a dopant with the purpose of changing the electrically conductive properties of the matrix material.

In some embodiments, the blend of matrix material and crystalline framework material may comprise further components such as conventional additives including dopants, and charge transport compounds (such as solid or liquid electrolytes). Furthermore, in some embodiments, laser dyes may be included as optional additives for changing or tuning the wavelength of the emitted light, wherein the laser dye can be physically separate from the electroluminescent compound. A non-limiting example of a laser dye that may be used for this purpose is Coumarin 540A.

In some embodiments, in a first method of producing the oriented film 20, a MOF is first synthesized and later deposited onto the surface of the substrate 18. In this non-limiting example, the as-synthesized MOF material includes a dispersion of crystals 22 having high aspect ratios such that the crystals are much larger in two dimensions than in a third. In this non-limiting example, each of the crystals 22 has both a length and width much greater than its thickness. In some cases, the crystals 22 have an aspect ratio of at least 5:1. In some cases, an aspect ratio greater than 10:1, and in some cases, at least 100:1. As used herein, these aspect ratios mean that the longest dimension of the crystal is at least 5, 10 or 100 times longer than the shortest dimension of the crystal in some embodiments.

The crystals 22 are deposited on the surface of the substrate 18 by any one of a number of methods including dip coating, spray coating, drop casting, and spin coating. The sheet-like crystals 22 are, in some cases, oriented parallel to the surface of the substrate 18. The film 20 deposited on the surface of the substrate 18, in some cases, comprises a coating of sheet-like MOF crystallites 22 whose orientation is substantially parallel to the surface of the substrate 18.

In some embodiments, the crystallites 22 have both a length and a width in the range of 50 to 10,000 nm (the horizontal directions in FIG. 3) and a thickness in the range of 5 to 100 nm (in the vertical direction in FIG. 3). In another method of fabrication, the crystalline framework material is synthesized directly onto the surface of the substrate 18.

In some cases, the crystallites or crystals 22 orient the transition dipoles of the luminescent emitters such that when the sheet-like crystals lie substantially flat on the surface of the substrate 18, the transition dipoles are parallel to the surface of the substrate and bottom surface of the film 20. In other embodiments, the high aspect ratio crystals are shaped like needles that are standing on end with their transition dipoles pointing perpendicular to the needle direction (longitudinal axis of the needle), and still parallel to the surface of the substrate 18. These shapes (sheets and needles) and other high aspect ratios shapes or morphologies are possible in alternative embodiments.

Note that the shapes and dimensions of the crystallites or crystals 22 are not critical to achieve the desired orientation of the transition dipoles and can be any shape or dimension. In some cases, the crystallites or crystals are flat sheet-like crystals 22 or upright needle-like crystals, or other exotic crystal geometries, as long as it keeps the relevant crystal axis oriented in the right way (e.g., parallel to the planar bottom surface of the film 20 and top surface of the substrate 18) so that the crystals 22 and their unit cells have a particular orientation with respect to the planar surface. In some embodiments, the crystallites or crystals are a cylindrical shape, a circular shape, a square shape, a spherical shape, a cone-shape, a prism-shape, or a rectangular shape. In some embodiments, each of the crystallites or crystals is the same shape. In some embodiments, each of the crystallites or crystals is a different shape. The crystallites or crystals are not limited to the shapes and/or sizes as described herein and can be any shape and/or size as required per conditions specified to its intended use.

In general, the crystals 22 form on the planar surface of the substrate 18 such that some crystallographic axis of the crystal structure is aligned substantially parallel to the planar surface of the film and substrate 18. That crystallographic axis could be the [001], the [011], the [111], the [020], the [435], etc., depending upon the specific MOF or other crystalline framework material that is used in that application. Once we identify which crystallographic axis of a given MOF material forms with a particular orientation that is substantially parallel to the planar surface of the film and substrate, we can incorporate the luminescent emitters into the MOF material with the correct positioning so that their transition dipoles are also oriented as desired (e.g., parallel to the crystallographic axis). In particular, the unit cells of the crystals 22 hold the luminescent emitters in the correct orientation so that their transition dipoles are substantially parallel to the crystallographic axis, which is in turn substantially parallel to the planar surface of the film and substrate 18. The crystals 22 are oriented such that the axis in question, i.e. the crystallographic axis to which the transition dipoles are held substantially parallel by the unit cells, has a particular orientation that is substantially parallel to the surface of the substrate 18, such that the transition dipoles have an anisotropy factor Θ less than ⅓.

Not all the crystals 22 need to be oriented perfectly parallel, nor does their selected crystallographic axis (to which the transition dipoles are aligned) need to be oriented perfectly parallel to the planar surface of the film and substrate 18. In some cases, most of the crystals or crystallites 22 will have an orientation that is closer to the ideal than random. We can specify that a “non-random orientation” is when an element or elements such as a crystallographic axis, the luminescent emitters, or their transition dipoles exhibit an average orientation that is different from the average orientation they would have if the orientation were random. The desired anisotropic orientation of the transition dipoles can still be achieved if at least some of the crystals 22 and their respective crystallographic axes (to which the transition dipoles are aligned parallel) are oriented parallel to the planar bottom surface of the film 20, with a maximum deviation of +/−45°, or in other embodiments+/−40°, +/−35°, +/−30°, +/−25°, +/−20°, or +/−15°. In some cases, at least 30%, and in some cases, at least 40%, 50%, 66%, 70%, 80%, 90% or 95% and at most 100% of all the crystals 22 and their respective crystallographic axes (to which the transition dipoles are aligned parallel) are oriented parallel to the planar surface with a maximum deviation of up to +/−45° or +/−40°, +/−35°+/−30°, +/−25°, +/−20°, or +/−15° from this parallel orientation.

In some embodiments, the plurality of luminescent emitters are configured to emit light from the planar surface of the film at an average angle of emission of 450 or less (e.g., 400 or less, 350 or less, 300 or less, 250 or less, 200 or less, 150 or less, or 100 or less) from normal.

In some embodiments, there may be more than one crystallographic axis of the crystal structure that is substantially parallel to the planar surface, and the transition dipoles are oriented substantially parallel to at least one of the substantially parallel crystallographic axes. In some embodiments, there may be just one crystal forming the film or emission layer, rather than the dispersion of multiple crystals 22.

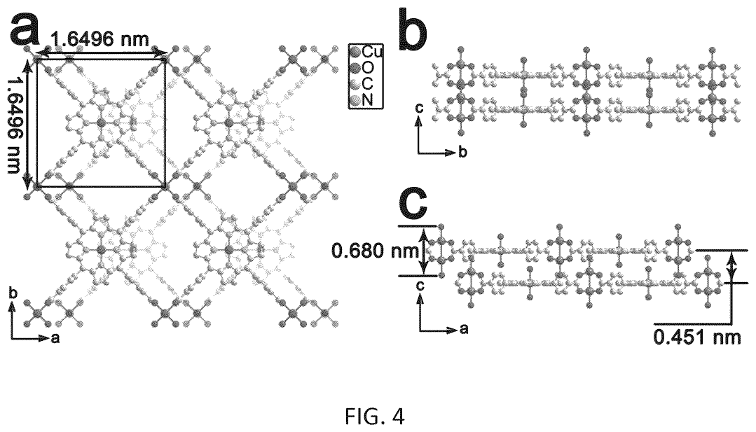

FIGS. 4A, 4B and 4C show the crystal structure of the Cu₂(TCPP) MOF. The TCPP unit, in which a platinum ion is bound at the center of the porphyrin moiety, is complexed to eight copper ions, which form (Cu₂O₈C₄) clusters referred to as “paddlewheel” structural units. In this embodiment, the chemical formula of the MOF is Cu₂(TCPP) where TCPP is 5,10,15,20-tetrakis(4-carboxyphenyl)porphyrin]. Cu₂(TCPP) may include any number of water or other solvent molecules within its chemical formula. In some embodiments, the emission layer comprises Cu₂(TCPP) and at least one conductive matrix element to facilitate charge transport to the luminescent moieties. In yet another embodiment, the pores of the Cu₂(TCPP) MOF are occupied by conductive organic molecules that facilitate charge transport to the luminescent moieties.

Referring again to FIG. 3, in a particular embodiment, a film of Cu₂(TCPP) is deposited by drop casting a dispersion of Cu₂(TCPP) in an alcohol solvent mixed with water onto a planar surface of the substrate 18. The sheet-like geometry of the Cu₂(TCPP) MOF results in a particular parallel orientation of the sheet-like crystallites 22 with respect to the planar surface of the substrate 18. The luminescent moiety TCPP of the Cu₂(TCPP) MOF is oriented with a particular parallel orientation with respect to the planar bottom surface of the film 20 and the planar top surface of the substrate 18. The transition dipole moment of TCPP lies in the plane of the MOF crystal, also in parallel orientation with respect to the planar bottom surface of the film 20 and the planar top surface of the substrate 18. This parallel orientation of the transition dipole moments results in light emission that is, in some cases, directed perpendicularly (vertically downward in FIG. 3) to the plane defined by the bottom surface of the film 20 and the planar surface of the substrate 18.

### Attachment of Luminescent Emitters to the Crystalline Framework

Aspects of the present disclosure include the crystalline framework material forming the film 20 and comprising one or more of the luminescent moieties as a building unit of the framework material (e.g., at least one luminescent moiety is a component of the unit cell of the crystalline framework material). In some embodiments, the crystalline framework material is a metal-organic framework comprising metallic structural units linked by organic structural units, and the luminescent moiety is comprised in at least a part of the organic structural units. In some embodiments, the luminescent moieties are covalently attached to at least a portion of the crystalline framework material. In other embodiments, the luminescent moieties reside within the pores of the crystalline framework material. In the context of the present disclosure, the term “pore” refers to any kind of opening in the crystalline framework that contributes to the framework's porosity.

In some embodiments of the present disclosure, existing luminescent emitters (e.g., luminophores or luminescent moieties) may be incorporated into MOF structures using a direct incorporation method, a covalent or coordination bonding method, a non-covalent, or non-coordinative incorporation method; however, we are not limited to such approaches and modifications.

In some embodiments, existing luminescent emitters are incorporated using a direct incorporation method. In some cases, the luminophore may be modified by addition of a plurality of coordinating groups that allow the luminophore to act as a structural organic component in the MOF unit cell. In some cases, modifications include, but are not limited to: addition of one or more carboxylic acid group, one or more alcohol group, one or more amine group, one or more imidazolyl group, or one or more pyridyl group to the organic ligands of the luminophore or by conversion of an existing aromatic to include a heteroatom such as a nitrogen atom. Any of these modifications may be pursued in conjunction with any other in a single luminophore or luminescent moiety.

In some embodiments, existing luminescent emitters are incorporated using a covalent or coordination bonding method. In some cases, the luminophore may be modified by addition of a plurality of functional groups to allow the luminophore to bind to the MOF structure through a plurality of covalent and coordination bonds. Modifications include, but are not limited to, those listed above as well as the addition of one or more coordinating moieties, such as a thiol group, a cyano group, or an isocyano group, or one or more moieties allowing for covalent attachment to a MOF unit cell, such as a ketone, an aldehyde, an azide group, an alkene group, and alkyne group, an epoxide group, or an organic halide.

In some embodiments, existing luminescent emitters are incorporated using a non-covalent, non-coordinative incorporation method into the MOF pore. In some cases, the luminophore may be included within the MOF by taking advantage of specific or non-specific interactions between the luminophore and the MOF pore, such as van der Waals interactions, quadrupole interactions, and dipole interactions. In some cases, a luminophore may be modified by addition of chemical moieties to increase the strength of these modes of interaction, for example by addition of alkyl, aromatic, or halide groups. In some cases, a luminophore may be selected for inclusion without chemical modification based on the strength and specificity of its interaction with a particular MOF pore.

In some aspects, a method for preparing the crystalline framework material forming the film or light-emission layer comprises the steps of (i) preparing a metal-organic framework using an organic structural unit with a functional group, and (ii) reacting the functional group with one or more luminescent moieties. These two steps can be performed in any order. The preparation of metal-organic frameworks is well-known in the art (see, for example, Rowsell et al, Microporous and Mesoporous Materials 2004, 73, 3-14). Typically, the preparation of a metal-organic framework involves heating a mixture containing inorganic salts and organic compounds in a specific solvent such as DMF at a specific temperature such as in the range of 60° C. to 120° C., for several hours to two days. Alternative preparations of metal-organic frameworks include mechano-chemical grinding, electrochemical synthesis, sonochemical synthesis, and microwave-assisted synthesis.

Providing an organic structural unit with a functional group is known in the art of organic chemistry. It is also possible to prepare the functionalized metal-organic framework by using a compound that already has a functional group as starting material for the organic structural unit. Step (i) of the method typically involves a preparation of a metal-organic framework using an organic structural unit that already comprises a functional group. Alternatively, the functional group may be introduced after having prepared the metal-organic framework.

An alternative method for preparing the crystalline framework material comprises the steps of (i) preparing a metal-organic framework using an organic structural unit; and (ii) adsorbing a luminescent emitter at the internal surface of the metal-organic framework. The luminescent emitter may also be adsorbed to one of the building blocks before it is used to prepare a metal-organic framework. In accordance with this alternative method, the luminescent emitter is not chemically bonded to the metal-organic framework, but instead adsorbed to the surface of the framework structure, and in some cases, to the internal surface of pores of the framework structure. Adsorption can, for instance, be performed from the gas phase, or from solution.

Crystalline framework materials in which the luminescent emitter is attached to coordinatively unsaturated metal sites in the metal-organic framework, can for example be prepared by impregnation, such as by dry impregnation (also known as incipient wetness or pore volume impregnation) using an amount of solution equal to the pore volume, or by wet impregnation, using an excess of solution volume. They can also be prepared by chemical vapor deposition or atomic layer deposition methodologies.

In some embodiments of crystalline framework materials wherein the luminescent moieties are chemically bonded to the organic structural units, the metal-organic framework comprises luminescent organic structural units that are the reaction product of (i) an organic structural unit provided with a functional group, and (ii) one or more luminescent moieties chosen from the group consisting of inorganic compounds, inorganic-organic compounds such as metal-organic compounds and organic compounds. As used herein and in its conventional sense, the term “functional group” refers to its usual and ordinary meaning in organic chemistry, namely an interconnected group of atoms that is responsible for a characteristic chemical reaction of the molecule to which the group is bonded. A functional group may comprise a leaving group.

The preparation of such luminescent organic structural units can be done by using various functional groups. For instance, the organic structural unit can be modified with one or more functional groups selected from the group consisting of amines, nitro groups, imines, pyridyls or derivatives thereof, haloformyls, haloalkyls, halogens including acyl halides such as acid chlorides. Good results have been achieved by modifying the organic structural unit with an amine functional group, but other functional groups may be used as well.

In some embodiments, a functional group may be used to couple an inorganic compound, an inorganic-organic compound such as a metal-organic compound and/or an organic compound to an organic structural unit so as to produce a luminescent organic structural unit. The functional group may comprise a leaving group, which may be replaced with the inorganic compound, the inorganic-organic compound, or the organic compound. Non-limiting examples of inorganic compounds, inorganic-organic compounds, and organic compounds suitable for use as luminescent moiety are organic laser dyes and compounds that comprise a phosphorus atom, such as phosphine and phosphinates. Non-limiting examples of metal-organic compounds suitable for use as luminescent moiety are compounds that comprise silver, gold, a rare earth element such as a lanthanide, or a ferrocene.

Note that not all of the unit cells in a film or emission layer of a device need to include a luminescent emitter. The film or emission layer will still emit some light in a desired direction, even when less than half of the unit cells include a luminescent emitter. When the luminescent emitter is chemically bonded to the organic structural units of the unit cells of the crystalline framework material (e.g., metal-organic framework) or resides in a pore, 30% or more of the unit cells may be functionalized with the luminescent emitter (e.g., a luminophore molecule or luminescent moiety). In some embodiments 50% or more, or 60% or more, or 70% or more of the unit cells may be functionalized with a luminescent emitter. In other non-limiting examples, 70% to 80% or 70% to 90% of the unit cells may be functionalized with a luminescent emitter. It may also be possible that substantially all of the unit cells of the crystalline framework material have been functionalized with a luminescent emitter.

In some embodiments, each luminescent emitter is covalently attached to a structural unit of the crystalline framework material via a pendant linker (e.g., a pendant luminescent emitter that is covalently linked to, but not part of, the core structure of the crystalline framework). In some embodiments, at least 50% of the organic structural units comprise a luminescent emitter having a transition dipole moment configured to be substantially parallel to the planar surface of the film. In some embodiments, the organic structural unit is composed of a luminescent emitter linked to one or more metal binding groups (e.g., two or more) up to six metal binding groups (e.g., up to 5, 4 or 3 metal binding groups). The metal binding groups can be attached to a luminescent emitter via any convenient locations, with any convenient spacing, to provide for a desired orientation of the luminescent emitter in the crystalline framework material.

In some embodiments, the organic structural unit of the present disclosure is of the formula:

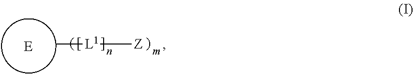

wherein E is the luminescent emitter; each L¹ is independently a linking group wherein each n is independently 0 or 1; Z is a metal-binding functional group; and m is 1 to 4.

Metal-binding functional groups of interest that can be included in the organic structural units of the MOFs described herein include, but are not limited to, —CO₂H, —CS₂H, —NO₂, —SO₃H, —Si(OH)₃, or a salt form thereof.

In some embodiments of formula (I), n is 0 and Z is directed covalently bound to E. In some embodiments of formula (I), n is 1 and the linker L¹ is present.

In some embodiments of formula (I), each L¹ is independently selected from alkyl, substituted alkyl, alkenyl, substituted alkenyl, alkynyl, substituted alkynyl, aryl, substituted aryl, heteroaryl, substituted heteroaryl, oxo (—O—), amido (e.g., —NHCO— or —CONH—), sulfonyl, sulfonamido, and the like. In some embodiments, L¹ is a macrocycle. In some embodiments, the macrocycle is selected from porphyrins and polydentate ligands. In some embodiments, the polydentate ligands are selected from bipyridine, phenylpyridine, salen ligands, and substituted versions thereof. In some cases, polydentate ligands are selected from terephthalate acid, amino-terephthalic acid, and nitro-terephthalic acid. Non-limiting examples of porphyrins and polydentate ligands can be found in U.S. Pat. Nos. 9,880,137 and 10,413,858, which are hereby incorporated by reference in their entirety.

In some embodiments of formula (I), m is 2, 3 or 4. In some embodiments of formula (I), m is 2. In some embodiments of formula (I), m is 3. In some embodiments of formula (I), m is 4.

In some embodiments of formula (I), the organic structural unit of the present disclosure is of one of the following the formulae:

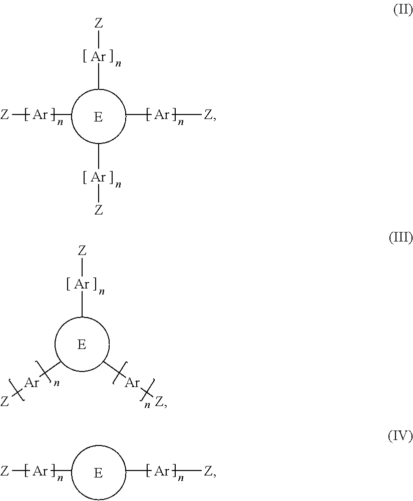

wherein:
each n is independently 0 or 1; and
each Ar is independently selected from aryl, substituted aryl, heteroaryl, substituted heteroaryl, heterocycle, and substituted heterocycle.

In some embodiments of formula (II)-(IV), Ar is phenyl or substituted phenyl. In some embodiments of formula (II)-(IV), Ar is 1,4-phenylene or substituted 1,4-phenylene. In some embodiments of formula (II)-(IV), each n is 1. In some embodiments of formula (II)-(IV), each n is 0 and the Z groups are directly covalent bound to E.

In some embodiments of formula (I)-(IV), each Z is carboxy.

In some embodiments, the luminescent emitter E is selected from iridium complexes, platinum complex, rare earth metal complexes, phenyl-based phosphines, phosphine oxides, and substituted phosphines. In some embodiments, E is a luminescent iridium complex. In some embodiments, E is a metal complex, e.g., a luminescent iridium complex, that includes multiple ligands (e.g., bidentate ligands) coordinated to a metal ion. In some embodiments of formula (II)-(IV), E is a metal complex, and the metal-binding functional groups are linked to just one of the ligands of the complex. In some embodiments of formula (II)-(IV), E is a metal complex, and the metal-binding functional groups are linked to two of the ligands of the complex. In some embodiments of formula (II)-(IV), E is a metal complex, and each metal-binding functional group is linked to a different ligand of the complex. In some cases, the geometry and configuration of coordinated ligands around a luminescent metal complex (e.g., an octahedral or other geometry) provides for linking positions or substitution positions which can be utilized to project metal-binding functional groups into a desirable orientation for connection to the crystalline framework (see e.g., FIG. 3).

In some embodiments of formula (III)-(IV), the organic structural unit of the present disclosure is of one of the following the formulae:

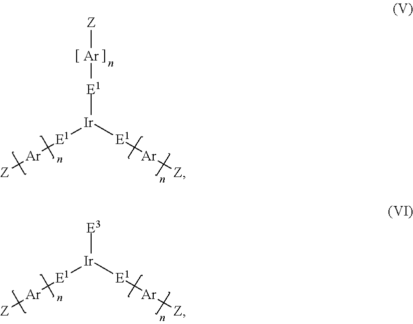

wherein E₁, E₂ and E₃ are each independently a bidentate chelating ligand of the luminescent iridium complex.

In some embodiments of formula (V)-(VI), E₁, E₂ and E₃ are independently selected from bppo, ppy, MDQ, acac, and substituted versions thereof. In some embodiments, E₁, E₂ and E₃ can be independently selected from substituted bppo, substituted ppy, substituted MDQ, and substituted acac. In some embodiments, a combination of E₁, E₂ and E₃ are selected such that E is (bppo)₂Ir(acac), (bppo)₂Ir(ppy), (ppy)₂Ir(bppo), (VIDQ)₂Ir(acac), or substituted versions thereof. E₁, E₂ and E₃ can be substituted at any convenient positions of the ligand with —[Ar]ₙ—Z, e.g., at a position of the ligand that does not adversely affect coordination of the ligand with the iridium ion, and which position is suitable for an alignment of the organic structural unit with the axes of the framework.

In some embodiments, each of the luminescent emitters is covalently attached to an organic structural unit via a pendant linker. In some embodiments, the organic structural unit of the present disclosure is of one of the formulae:

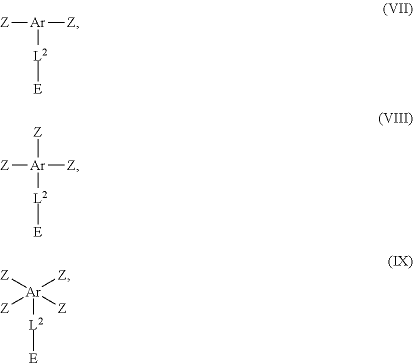

wherein: E is the luminescent emitter; Ar is selected from aryl, substituted aryl, heteroaryl and substituted heteroaryl; L² is a pendant linker; and Z is a metal-binding functional group. Ar can be monocyclic, or a multicyclic ring system.

In some embodiments of formula (VII)-(IX), Ar is selected from porphyrin, perylene, pyridine, phenyl, imidazole, bipyridine, triazole, and substituted versions thereof. In some embodiments, Ar is phenyl or substituted phenyl. In some embodiments, Ar is phthalic acid, isophthalic acid, terephthalic acid, trimesic acid, or a substituted version thereof.

In some embodiments of formula (VII), the organic structural unit of the present disclosure is of the formula:

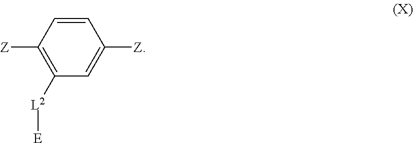

In some embodiments of formula (VII)-(X), Z is carboxy. In some embodiments of formula (VII)-(X), the luminescent emitter E is selected from iridium complexes, platinum complex, rare earth metal complexes, organic dyes, phenyl-based phosphines, phosphine oxides, substituted phosphines.

In some embodiments of formula (VII)-(X), L² is independently selected from alkyl, substituted alkyl, alkenyl, substituted alkenyl, alkynyl, substituted alkynyl, aryl, substituted aryl, heteroaryl, substituted heteroaryl, oxo (—O—), amino, substituted amino, amido (e.g., —NHCO— or —CONH—), sulfonyl, sulfonamide, etc. In some embodiments of formula (VII)-(X), L² comprises a substituted amino group. In some cases, L² further comprises a metal-binding ligand group capable of coordination to a luminescent metal complex.

In some embodiments of formula (VII)-(X), L² is — NR—P(R′)(R″)═O— and is coordinated to a luminescent metal complex. In some embodiments, R, R′ and R″ are independently selected from hydrogen, alkyl, alkenyl, alkynyl, aryl, heteroaryl, amino, alkoxy, hydroxy, aryloxy, heteroaryloxy, heterocyclic oxy group, acyl, alkoxycarbonyl, aryloxycarbonyl, acyloxy, acylamino, alkoxycarbonylamino, aryloxycarbonylamino, sulfonylamino, sulfamoyl, carbamoyl, alkylthio, arylthio, heterocyclic thio group and a heterocyclic group. In some embodiments, R is hydrogen or alkyl; and R′ and R″ are independently selected from alkyl, aryl, heteroaryl, alkoxy, hydroxy, aryloxy, heteroaryloxy, and acyloxy.

In some embodiments of formula (VII)-(X), the organic structural unit of the present disclosure is of the formula:

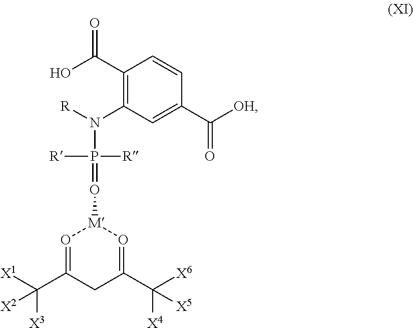

wherein: M′ is a metal ion; and X¹, X², X³, X⁴, X⁵, and X⁶ are each independently hydrogen or halogen atom; or a salt form thereof.

In some embodiments of formula (XI), X¹, X², X³, X⁴, X⁵, and X⁶ are each hydrogen.

In some embodiments of formula (XI), M′ is a rare earth metal ion. In some embodiments, the rare earth metal ion is selected from yttrium, lanthanum, cerium, praseodymium, neodymium, europium, gadolinium, terbium, dysprosium, holmium, erbium, thulium, ytterbium, and lutetium.

In a non-limiting example of the crystalline framework material forming the film or light-emission layer, at least some of the organic structural units of the metal-organic framework have a pendant functional group in the form of an amino group that is connected to a core structural unit, such as a phthalic acid, such as isophthalic acid, or terephthalic acid. It has been found that such pendant functionalized amino groups can be readily linked or reacted with a suitable luminescent moiety. Non-limiting examples of resulting luminescent organic structural units are those of formula (XII) and formula (XIII) below.

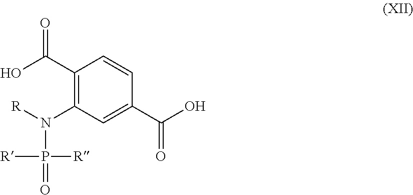

In formula (XII), R can be hydrogen, an alkyl group, an alkenyl group, an alkynyl group, an aryl group, an amino group, an alkoxy group, an aryloxy group, a heterocyclic oxy group, an acyl group, an alkoxycarbonyl group, an aryloxycarbonyl group, an acyloxy group, an acylamino group, an alkoxycarbonylamino group, an aryloxycarbonylamino group, a sulfonylamino group, a sulfamoyl group, a carbamoyl group, an alkylthio group, an arylthio group, a heterocyclic thio group or a heterocyclic group. R′ can be the same as R″ and can be a phenyl ring which may optionally be substituted.

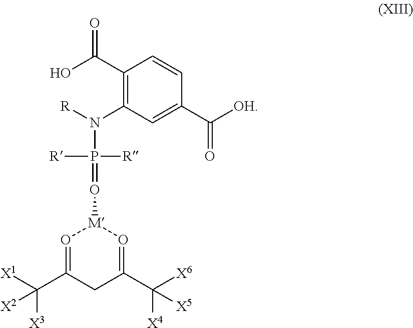

In some embodiments, in general formula (XIII), R can be hydrogen, an alkyl group, an alkenyl group, an alkynyl group, an aryl group, an amino group, an alkoxy group, an aryloxy group, a heterocyclic oxy group, an acyl group, an alkoxycarbonyl group, an aryloxycarbonyl group, an acyloxy group, an acylamino group, an alkoxycarbonylamino group, an aryloxycarbonylamino group, a sulfonylamino group, a sulfamoyl group, a carbamoyl group, an alkylthio group, an arylthio group, a heterocyclic thio group or a heterocyclic group. R′ can be the same as R″ and can be a phenyl ring which may optionally be substituted. M′ is a metal ion, for example a rare earth metal ion, such as a rare earth metal ion selected from the group consisting of yttrium, lanthanum, cerium, praseodymium, neodymium, europium, gadolinium, terbium, dysprosium, holmium, erbium, thulium, ytterbium, and lutetium. X¹, X² X³, X⁴, X⁵, and X⁶ can each independently be a halogen atom, such as fluorine, chlorine, or bromine. X¹, X², X³, X⁴, X⁵, and X⁶ can be the same.

The luminescent emitter may also be configured within pores of the crystalline framework material. In some embodiments, each of the luminescent emitters is non-covalently bound to an organic structural unit and configured in a pore of the metal-organic framework. The luminescent emitter can, for example, be adsorbed to the inner surface of a metal-organic framework. The luminescent emitter can be intercalated after preparation of the metal-organic framework via known methods such as vapor deposition and/or adsorption and/or post-synthetic functionalization. It is also possible to prepare the metal-organic framework in the presence of a luminescent moiety which will then be incorporated (encapsulated or embedded) in the porous structure of the metal-organic framework. The close proximity and regular organization of the luminescent moieties may lead to the occurrence of charge (electron) hopping mechanisms. Furthermore, luminescent moieties can be used that lack a reactive group thereby extending the range of possible luminescent moieties that can be used in the crystalline framework material. When the luminescent emitter resides in pores of the metal-organic framework, the latter may be loaded with the luminescent emitter to an extent of at least 0.3 to 0.8 luminescent moieties per organic structural unit of the metal-organic framework.

### Luminescent Moieties

A variety of luminescent emitters can be adapted for use in the subject films. The term “luminescent moiety” refers to a moiety or compound which is capable of emitting light. The luminescent moiety may be photoluminescent or electroluminescent and capable on excitation (e.g., by light or application of a potential difference) of emitting light. The emission of light can occur because of an excitation in the moiety or compound and can have different forms including but not limited to photoluminescence, fluorescence, phosphorescence, electroluminescence, thermoluminescence, etc. The terms “luminescent moiety”, “luminescent emitter”, and “luminophore” are used interchangeably herein. In some cases, a luminescent moiety is a phosphor.

In some embodiments, luminescent emitters of the present disclosure are selected from iridium complexes, platinum complexes, rare earth metal complexes, organic dyes, phenyl-based phosphines, phosphine oxides, substituted phosphines, and the like. Combinations of two or more of any of the luminescent emitters described herein can be utilized to provide desired light emission properties from the subject films.

In some embodiments, the luminescent emitters are luminescent transition metal complexes. In some embodiments, the luminescent emitters are iridium complexes. In some embodiments, the luminescent emitters are rhenium complexes. In some embodiments, the luminescent emitters are platinum complexes.

In some embodiments, the transition metal complex can have the formula:

L¹¹L¹²L¹³M

wherein:

M is a transition metal ion; and

L¹¹, L¹² and L¹³ are organic bidentate ligands coordinated to M.

In some embodiments, M is iridium (Ir). In some embodiments, M is rhenium (Re). In some embodiments, M is Pt(IV).

Iridium complexes of interest including organic bidentate ligands that can be adapted for use in the subject films include, but are not limited to, those iridium complexes described in US20070292631, and U.S. Pat. No. 9,793,499, the disclosures of which are herein incorporated by reference.

In some embodiments, at least one of the ligands L¹¹, L¹² and L¹³ are is an organic bidentate ligand that coordinates M via a nitrogen atom and a carbon atom. In some embodiments, at least one of the ligands L¹¹, L¹² and L¹³ is a β-diketone ligand. Organic bidentate ligands of interest include β-diketone ligands (e.g., acac), ppy=2-phenylpyridine, ppz=4,7-phenanthrolino-5,6:5,6-pyrazine, bppz=2,3-di-2-pyridylpyrazine, bppo, MDQ (2-methyldibenzo[f,h]quinoxaline), and ppo (PPO=3-phenyl-2-(phenylsulfonyl)-1,2-oxaziridine).

In some embodiments, the iridium complex is represented by the formula:

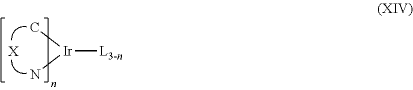

wherein X represents an atom group that forms an aromatic chelate ligand together with a carbon atom and a nitrogen atom bonded with iridium, n is an integer of 2 or 3, the multiple X's present in each aromatic chelate group may be the same or different from each other, and L represents a bidentate organic ligand wherein the atoms other than carbon atoms are bonded with iridium. In some embodiments, in the iridium complex above, the inside of [ ] bidentate ligand in the formula is any one of ligands selected from the group of structural formulas shown below:

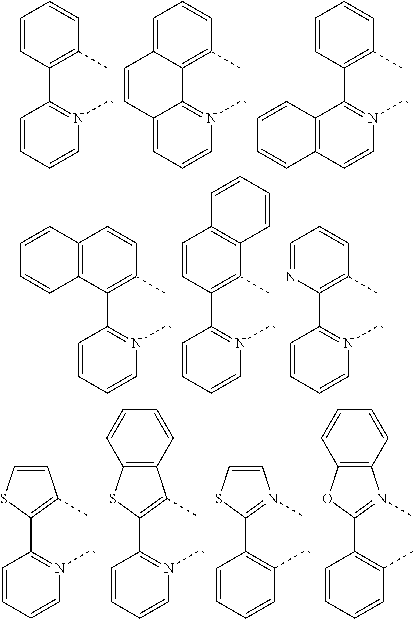

wherein a dotted line shows bonding with iridium, each hydrogen atom on the aromatic rings may be substituted by a halogen atom or an organic group having 1 to 15 carbon atoms, and when n represents a plural number, the ligands may be different from each other.

In some embodiments, a luminescent emitter is an organic iridium complex represented by the formula:

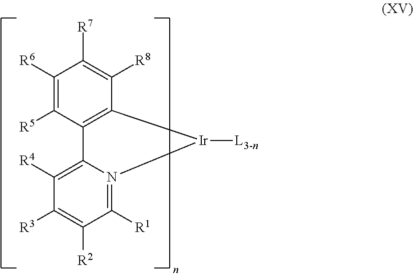

wherein R¹ through R⁸ independently represent a hydrogen atom, a halogen atom or an organic group having 1 to 15 carbon atoms, substituent groups (R¹ through R⁸) adjacent to each other may be bonded at one or more sites to thereby form a condensed ring, n is an integer of 2 or 3, two or three ligands shown by the inside of [ ] may be same or different from each other, and L represents a bidentate organic ligand, e.g., wherein the atoms other than carbon atom are bonded to iridium.

In some embodiments, a luminescent emitter is an organic iridium complex represented by the following Formula

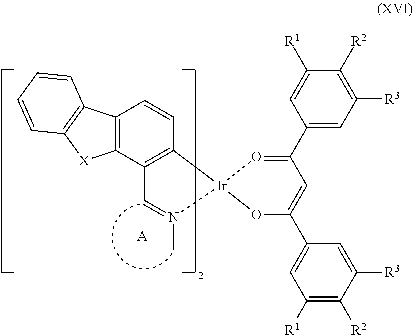

wherein R¹, R², and R³ are each independently a tert-butyl group or a hydrogen atom, and the β-diketone ligand has at least one tert-butyl group; they may bond each other to thereby form a saturated hydrocarbon ring when the β-diketone ligand has two tert-butyl groups; A is a substituent having a heterocyclic ring which is either a 5-membered ring or a 6-membered ring and containing nitrogen; the heterocyclic ring of A is optionally fused to a benzene ring and may include sulfur atom (S) or oxygen atom (O) as a hetero atom other than nitrogen (N); X is a hetero atom.

In some embodiments, the luminescent emitters are selected from tetraphenylporphyrin platinum(II), (bppo)₂Ir(acac), (bppo)₂Ir(ppy), (ppy)₂Ir(bppo), (ppy)Re(CO)₃, and (MDQ)₂Ir(acac), Ir(piq)3, (piq)₂Ir(acac), (pq)2Ir(acac), Ir(ssppy)3, Ir(pppy)3, tetraphenylporphyrin platinum(II), (bppo)2Ir(acac), (bppo)2Ir(ppy), (ppy)₂Ir(bppo), (ppy)Re(CO)3, (MDQ)₂Ir(acac), Flrpic, Flr6, Flrtaz, FlrN4, FCNlr, Ir(dfpypy)3, Ir(taz)3, mer-Ir(cn-pmic).

In some embodiments, the luminescent moieties are selected from the group consisting of rare earth metal complexes, organic dyes, phenyl-based phosphines, phosphine oxides, substituted phosphines containing hydrogen, alkyls, alkenyls, alkynyls, aryls, amines, alkoxides, aryloxides, heterocyclic oxides, acyls, alkoxycarbonyls, aryloxycarbonyls, acyloxides, acylamines, alkoxycarbonylamines, aryloxycarbonylamines, sulfonylamines, sulfamoyls, carbamoyls, alkylthiols, arylthiols, heterocyclic thiols, and combinations thereof.

In some embodiments, the luminescent moieties are organic dyes. Organic dyes of interest include, but are not limited to, eosin dyes, rhodamine dyes, xanthene dyes, fluorescein dyes, acridine dyes, anthraquinone dyes, azo dyes, diazonium dyes, fluorine dyes, fluorone dyes, phthalocyanine dyes, BODIPY dyes, and N,N-di(1-naphthyl)-N,N-diphenyl-(1,1′-biphenyl)-4,4′-diamine (NPD).

### Transition Dipole Moment Orientation

In some embodiments, the overall emission of a luminophore molecule or luminescent moiety can be described as a superposition of the contribution from horizontally and vertically aligned transition dipoles, where the orientation is taken with respect to the planar bottom surface of the film or emission layer 34, or with respect to a planar surface of the stack (e.g., the bottom surface of the transparent substrate 40 or whatever substrate is the bottom layer of the stack forming the OLED device).

In some embodiments, the luminescent film of the present disclosure is composed of a plurality of crystals composed of the crystalline framework material and each having at least one crystallographic axis aligned substantially parallel to the planar surface of the film, wherein a plurality of the repeating structural units of each crystal comprise a luminescent emitter having a transition dipole moment configured in a substantially parallel orientation relative to the at least one crystallographic axis. In some embodiments, the repeating structural units of the crystalline framework material each comprise a structurally integrated luminescent emitter (e.g., a luminescent emitter that is part of, and essential to, the structure of the crystalline framework). In some cases, at least 50% of the repeating structural units comprise a luminescent emitter having a transition dipole moment configured to be substantially parallel to the planar surface of the film. In some cases, at least 66% of the transition dipole moments of the luminescent emitters are oriented substantially parallel to the planar surface of the film with a maximum deviation of about +/−45°, about +/−40°, about +/−350, about +/−30°, about +/−25°, about +/−20°, about +/−15°, about +/−10°, or about +/−5°. In some embodiments, the luminescent emitters are oriented “substantially parallel” to the planar surface of the film with a maximum deviation of about 450 or less, about 400 or less, about 350 or less, about 300 or less, about 250 or less, about 200 or less, about 150 or less, about 100 or less, or about 5° or less, from this parallel orientation.

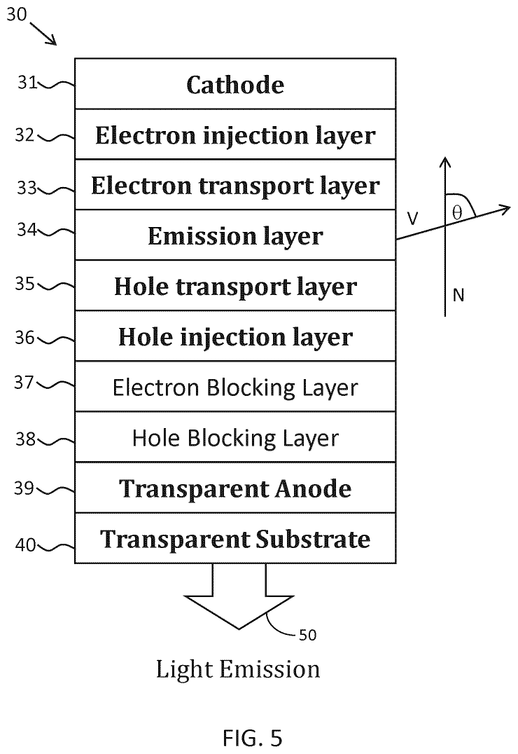

In the non-limiting example in FIG. 5, the desired parallel orientation of the transition dipoles is horizontal, and a perpendicular orientation of the dipoles is vertical. The anisotropy factor is the ratio of the number of vertical dipoles to the total number of dipoles and hence describes the average orientation of the transition dipole moment. The anisotropy factor Θ can be written as:

Θ=<cos²θ> (1)

where θ is the angle between a respective transition dipole moment vector V of a luminescent emitter in the emission layer 34 and a surface normal N, wherein the surface normal N is perpendicular to the planar surface of the emission layer 34. Isotropic orientation (Θ=⅓) is present if ⅓ of the transition dipole moments are aligned perpendicular to the planar surface of the emission layer 34 (e.g., vertically oriented), and ⅔ are horizontally oriented. Optical simulations have shown that maximum outcoupling efficiency is achieved for Θ=0, in other words complete parallel (e.g., horizontal) alignment of all transition dipole moments. Completely horizontally aligned transition dipoles result in Θ=0. An isotropic distribution is given by Θ=0.33, and vertical dipole orientation is indicated by Θ=1. In some embodiments, the anisotropy factor θ is less than 0.2; or less than 0.1; or less than 0.015; or less than 0.001 or, in some cases, 0.

In some cases, the luminescent emitters are configured to be substantially uniformly oriented relative to the planar surface of the film and have an orientation anisotropy factor (Θ) of less than 0.33. An orientation anisotropy factor less than 0.33 indicates that the transition dipoles in the film of emission layer 34 are, in some cases, oriented parallel to the planar surface of the emission layer to emit a greater proportion of light in a perpendicular direction to the planar surface than would be the case for luminescent moieties with random orientation. The planar surface of the film or emission layer 34, to which the luminescent moieties and their transition dipoles have a parallel orientation, and through which light is emitted, is typically a major surface (e.g., the top or bottom of the film or emission layer, usually in contact with a substrate surface), not a minor edge surface of the film or emission layer.

In some embodiments, not all of the luminescent emitters and their respective transition dipoles in the film or emission layer 34 need to be oriented perfectly parallel to the planar surface. The desired anisotropic orientation can still be achieved if at least some of transition dipoles are oriented substantially close to parallel. In some embodiments, the transition dipole moments may be arranged parallel (horizontal in FIG. 5) with respect to the planar bottom surface of the film, emission layer 34, or planar surface of an OLED stack with a maximum deviation of +/−45°, or in other embodiments+/−40°, +/−35°, +/−30°, +/−25°, +/−20°, or +/−15°. In some cases, at least 30%, 40%, 50%, 66%, 70%, 80%, 90% or 95% and at most 100% of all the transition dipoles are oriented parallel with a maximum deviation of up to +/−45° or +/−40°, +/−35°, +/−30°, +/−25°, +/−20°, or +/−15° from this parallel orientation.

In some embodiments, the light-emitting device 30 may also include a controller or voltage source for applying a voltage (potential difference) between the first and second electrodes. In some embodiments, a controller is arranged to apply an AC voltage, for example an AC voltage having a frequency in a range between 100 Hz and 10,000 Hz. The lower limit of this frequency range is chosen to avoid flicker, and the upper limit to make optimal use of charge generation processes in the light-emitting module. The AC voltage, in some cases, has a waveform wherein the transition between minimum to maximum takes place in a relatively short period of time, because it has been experimentally determined that for such waveforms the light-emitting device has the best performance. Non-limiting examples of such waveforms are a square wave, a triangle wave, and a pulsed wave. The AC voltage may consist of negative or positive voltages only, by applying a DC bias on the AC signal. This has the advantage that only one type of amplifier can be used.

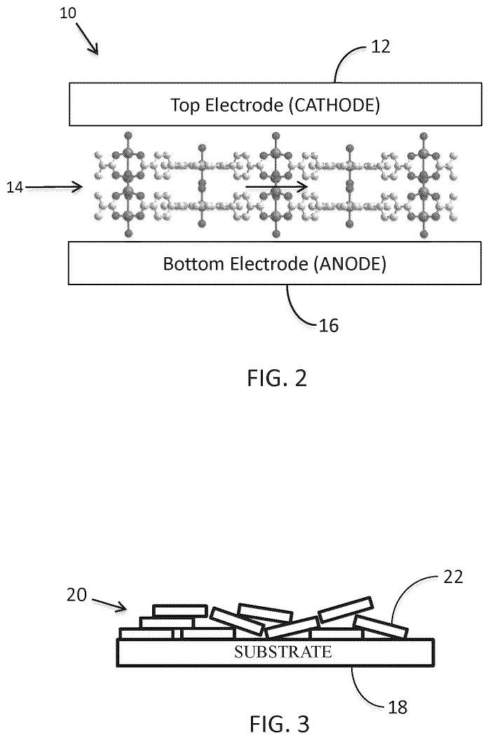

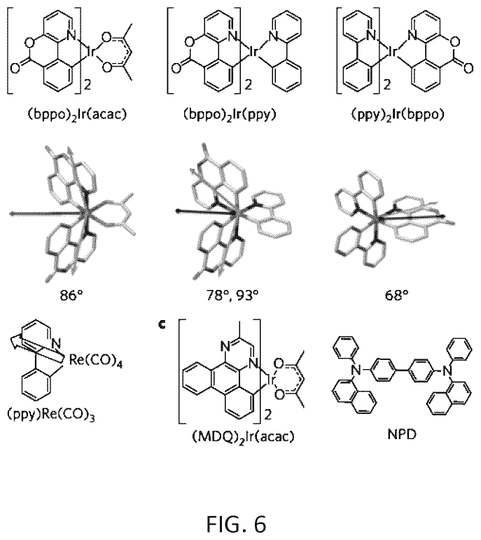

FIG. 6 shows some other common phosphor molecules with their transition dipoles indicated by arrows. Using similar substitution methods, these phosphors may be incorporated into similar MOF structures. For example, substituting carboxylic acid groups onto the ligands at particular positions leads to different orientations of the phosphor in the MOF unit cell, and thus to different orientations of the transition dipole moment. By selecting the positions for substitution, it is possible to control the orientation of the transition dipole moment of the phosphors in the final device. Note that the device in FIG. 2 is an example for which the transition dipole moment is pre-determined by the crystal structure of the MOF unit cells, whereas the phosphors in FIG. 6 may be designed in a variety of ways to orient the transition dipole moment as desired. Because the transition dipole (or multiple dipoles for some phosphor molecules) do not lay in the plane of the phosphor molecule, we can adjust the relative angle at which the phosphors are held in the MOF unit cell so that the transition dipoles are still parallel to the film surface through which light (e.g., photons) is emitted.

In some embodiments, adjustments to the structure of the MOF/phosphor unit cell are made until the transition dipole is as close to parallel to the film surface as possible. In some embodiments, a design process includes the steps of: A) identifying a nominal list of MOFs that can be grown or cast as a film on a planar surface of a substrate such that at least one crystallographic axis of the crystal structure is close to parallel to the planar surface of the substrate; B) learn what orientation the MOF unit cells are likely to have in the film (e.g., by making the MOF) and identifying which crystallographic axis grows substantially parallel; C) based on the MOF orientation in the film, determining what orientation the phosphor requires in the MOF unit cell for the dipole moment of the phosphor to be close to substantially parallel to the identified crystallographic axis; and D) putting carboxylate groups on the phosphor which allows incorporation of the phosphor into the MOF unit cell, wherein the carboxylate groups are arranged so that the phosphors and their transition dipoles will have the anisotropic orientation on average in the film.

Non-limiting examples of MOFs that may provide a suitable structure or film orientation include, but are not limited to: the class of MOFs based on zirconium oxide clusters, including UiO-66, UiO-66(NH₂), UiO-67, and UiO-68(NH₂). This class of MOFs is understood to include actual and hypothetical structures based on organic carboxylates connected to zirconium oxide clusters where the cluster contains at least six zirconium ions with coordination bonds to at least six organic carboxylates. In some cases, these MOFs are to be synthesized as a continuous film with the [111] crystal axis parallel to the substrate.

Non-limiting examples of MOFs that may provide a suitable structure or film orientation include, but are not limited to: the class of MOFs based on a “paddlewheel” M₂(R—CO2)₄ cluster where M is a divalent metal ion, most commonly Zn or Cu and organic dicarboxylate or tetracarboxylate moiety connecting two or four of these metal cluster units, respectively. In some cases, these coordination bonds result in a two dimensional sheet-like structure that extends along the [001] crystallographic axis. In some cases, these MOFs are to be synthesized as a continuous film with the [001] crystal axis parallel to the substrate.

Non-limiting examples of MOFs that may provide a suitable structure or film orientation include, but are not limited to: the class of MOFs based on a “paddlewheel” M₂(R—CO2)₄ cluster where M is a divalent metal ion, most commonly Zn or Cu and organic dicarboxylate or tetracarboxylate moiety connecting two or four of these metal cluster units, respectively, as well as an organic diamine or 4,4′-bipyridine moiety. In some cases, these coordination bonds result in a two dimensional sheet-like structure that extends along the [001] crystallographic axis interconnected by the carboxylate moieties. The sheets are interconnected perpendicular to the [001] axis by diamines bound to the metal ions in the axial position. In some cases, these MOFs are to be synthesized as a continuous film with the [001] crystal axis parallel to the substrate.

Non-limiting examples of MOFs that may provide a suitable structure or film orientation include, but are not limited to: the class of MOFs based on a “paddlewheel” M₂(R—CO2)₄ cluster where M is a divalent metal ion, most commonly Zn or Cu and organic tricarboxylate moiety connecting three of these metal cluster units into an interconnected three dimensional structure. HKUST-1 is an example of such a structure. In some embodiments, these MOFs are to be synthesized as a continuous film with either the [001] or the [111] crystal axes parallel to the substrate, depending on synthesis conditions and surface preparation.

Non-limiting examples of MOFs that may provide a suitable structure or film orientation include, but are not limited to: the class of MOFs based on divalent metal cations, typically Zn or Cd, and imidazolate moieties, collectively termed “zeolitic imidazolate frameworks.” ZIF-8 is an example of such a structure. In some embodiments, these MOFs are to be synthesized as a continuous film with a particular crystal axis parallel to the substrate.

### Light-Emitting Device

Aspects of the present disclosure include a light-emitting device, such as an organic light-emitting diode (OLED), and in particular to a light-emitting device and a crystalline film suitable for use in an emission layer of the device. In some embodiments, emitter materials, such as luminescent moieties or molecules, are maintained in a desired orientation by incorporating them into a crystalline framework material (e.g., a metal-organic framework or covalent organic framework).

In some cases, the luminescent film of the device is referred to as a luminescent thin film. In some embodiments, an emission layer of the present disclosure includes a luminescent film; and first and second electrodes between which a voltage can be applied to generate an electric field in at least part of the emission layer.

In some embodiments, at least one of the electrodes is composed of a transparent or semitransparent material. In some embodiments, the device includes one or more additional layers selected from an electron injection layer, electron transport layer, hole transport layer, hole injection layer, electron blocking layer, hole blocking layer and insulating layer.

In some embodiments, the device emits a visible light under an applied voltage.

Aspects of the present disclosure include an electronic apparatus comprising a light-emitting device of the present disclosure, wherein the electronic apparatus is selected from a video camera, a digital camera, a goggle type display, a navigation system, a personal computer, a portable information terminal, and the like.

FIG. 2 is a schematic diagram of a light-emitting device 10 comprising a top electrode (cathode) 12, a bottom electrode (anode) 16, and a MOF-containing emission layer 14 positioned between the cathode 12 and the anode 16. In some embodiments, the device 10 may optionally include several other layers between the cathode 12 and anode 16, as is discussed in alternative embodiments below. Such optional layers in an organic stack of an OLED device are well known in the art, but for simplicity of explanation are omitted from FIG. 2. The MOF-containing emission layer 14 is a MOF film having unit cells with a structure that is modified to include the phosphor molecule in FIG. 1 by substituting the tetraphenylporphyrin-copper unit with the phosphor molecule. The MOF film forming the emission layer 14 is a structural derivative with tetraphenylporphyrin platinum(II) (TPP-Pt).

The transition dipole moment of TPP-Pt is indicated by a horizontal arrow in FIG. 2 and lies at a predetermined angle parallel to the surface of the anode 16 (or parallel to the surface of the substrate on which the MOF film is deposited or grown if the device 10 includes additional layers between the emission layer 14 and the anode 16). The MOF film of the emission layer 14 orients the transition dipole moments of the phosphors parallel to its layered structure by design, i.e., parallel to a planar surface of the film and also parallel to the corresponding planar surface of the substrate on which the film is deposited or grown. The MOF is known to generate films that are highly oriented based on its layered structure.

The device 10 whose emission layer 14 comprises such a MOF film, has the transition dipoles oriented parallel to the surface of the emission layer 14 and parallel to the surface of the substrate on which the film is deposited or grown, which may be the transparent anode 16, or another substrate in the stack. Because the unit cells of the MOF film have a known orientation with respect to the device 10, and the phosphor has a known orientation within the MOF unit cell, the orientation of the phosphor transition dipoles with respect to the device 10 can be predicted prior to fabrication. The light (e.g., photons) is emitted perpendicular (vertically downward in FIG. 2) to the transition dipole moment (horizontal in FIG. 2), and thus the photons are emitted perpendicular to the planar bottom surface of the light-emitting layer 14 and perpendicular to the surface of the transparent anode 16. This results in greatly increased efficiency of the emission of light from the device 10. Far fewer photons are emitted parallel to the plane of these surfaces, which photons are lost to surface plasmon and other lossy modes. The arrangement of the light-emitting device 10 may result in up to a 50% increase in the photons that exit the device, and thus to an efficiency increase of up to 50%.

In some embodiments, the light-emitting device 10 may also include a controller or voltage source for applying a voltage (potential difference) between the first and second electrodes. In some cases, the controller is arranged to apply an AC voltage, for example an AC voltage having a frequency in a range between 100 Hz and 10,000 Hz. The lower limit of this frequency range is chosen to avoid flicker, and the upper limit to make optimal use of charge generation processes in the light-emitting module. The AC voltage, in some cases, has a waveform wherein the transition between minimum to maximum takes place in a relatively short period of time, because it has been experimentally determined that for such waveforms the light-emitting device has the best performance. Non-limiting examples of such waveforms are a square wave, a triangle wave, and a pulsed wave. The AC voltage may consist of negative or positive voltages only, by applying a DC bias on the AC signal. This has the advantage that only one type of amplifier can be used.

In alternative configurations of the light-emitting device 10, the first and second electrodes may both be provided at the same side of the light emission layer 14. In some embodiments, the light-generating layer may also be sandwiched between the first and second electrodes, wherein one of the electrodes is at least partially transparent. In some embodiments, transparent conductive oxides such as tin-doped indium oxide (also known as ITO) can be used as transparent electrode materials. In alternative embodiments, one or more of the electrodes may be translucent rather than transparent. As used herein and in its conventional sense, “translucent” may include a layer which is transmissive to visible light. In this case, the translucent layer can be transparent, that is to say clearly translucent, or at least partly light-scattering and/or partly light-absorbing, such that the translucent layer can, for example, also be diffusely or milkily translucent. In some cases, a layer designated here as translucent is embodied such that it is as transparent as possible, with the result that, in particular, the absorption of light is as low as possible.

Alternatively, in some cases, the light emission layer 14 may be sandwiched between the first and second electrodes 12 and 16, wherein the second electrode is located at an edge of the light-emission layer 14. In some embodiments, any conventional electrode materials can be used, such as aluminum or gold. The light-emitting device 10 may further comprise an auxiliary layer that is located between the light emission layer 14 and at least one of the first and second electrodes 12 and 16. An example of such an auxiliary layer is a dielectric layer, which can prevent direct interaction between the light emission layer 14 and the at least one of the first and second electrodes 12 and 16.

FIG. 5 shows a schematic block diagram of an electroluminescent device 30 having an emission layer 34 comprising a film composed of crystalline framework material, such as the oriented film of the previous embodiment. The device 30 includes a cathode 31 and a translucent or transparent anode 39 positioned on a device substrate 40 that also may be translucent or transparent. In addition to the emission layer 34 positioned between the cathode and anode, the device 30 includes many optional layers in a stack between the cathode and anode including: an electron injection layer 32, an electron transport layer 33 (may be multiple distinct layers), a hole transport layer 35 (may be multiple distinct layers), a hole injection layer 36, an electron blocking layer 37, and a hole blocking layer 38. The emission layer 34 comprises a MOF film with oriented luminescent moieties having transition dipoles oriented parallel to the planar bottom surface of the emission layer 34. This parallel orientation of the transition dipole moments results in the emission of light 50 (e.g., photons) that is, in some cases, directed perpendicularly (vertically downward in FIG. 5) to the plane defined by the bottom surface of the emission layer 34, through the transparent anode 39 and device substrate 40.

In the following description, it is understood that all recited connections between structures can be direct operative connections or indirect operative connections through intermediary structures. A set of elements includes one or more elements. In the claims, the word “comprising” does not exclude other elements, and the indefinite article “a” or “an” does not exclude a plurality. A plurality of elements includes at least two elements. Unless otherwise required, any described method steps need not be necessarily performed in a particular illustrated order.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A light-emitting device comprising an emission layer, and first and second electrodes between which a voltage can be applied to generate an electric field in at least part of the emission layer, wherein the emission layer comprises:
a) a crystalline framework material comprising a plurality of units cells having a crystal structure that is repeated throughout the crystalline framework material, wherein the crystalline framework material has a crystallographic orientation with respect to a planar surface of the emission layer so that a majority of the unit cells are substantially uniformly oriented with respect to the planar surface; and
b) a plurality of luminescent emitters incorporated into at least some of the unit cells, wherein the luminescent emitters are arranged in the unit cells such that the transition dipole moments of the luminescent emitters are oriented with respect to the planar surface of the emission layer such that their orientation anisotropy factor, Θ, is less than 0.33. In some cases, each of the unit cells that is functionalized with at least one of the luminescent emitters holds or positions the luminescent emitter in a substantially fixed orientation with respect to the crystal structure of the unit cell so that the transition dipole moment of the luminescent emitter is maintained in the desired orientation (e.g., substantially parallel to the planar surface).

Aspect 2. The device of aspect 1, wherein at least 50% of the unit cells hold at least one of the luminescent emitters in the desired orientation (e.g., so that the transition dipole is substantially parallel to the planar surface).

Aspect 3. The device of aspect 1, wherein at least 66% of the transition dipole moments are oriented substantially parallel to the planar surface of the emission layer with a maximum deviation of +/−45°.

Aspect 4. The device of aspect 1, wherein the emission layer comprises a film composed of one or more crystals comprising the unit cells of the crystalline framework material, each of the one or more crystals has at least one crystallographic axis that is aligned substantially parallel to a planar surface of the film, and the luminescent emitters are arranged in at least some of the unit cells (e.g., at least 30%) such that the transition dipole moments of the luminescent emitters are substantially parallel to the crystallographic axis.

Aspect 5. The device of aspect 1, wherein the crystalline framework material comprises at least one metal-organic framework including metallic structural units linked by organic structural units, and wherein the luminescent emitters are comprised in at least a part of the organic structural units.

Aspect 6. The device of aspect 1, wherein the luminescent emitters reside in pores of the crystalline framework material.

Aspect 7. The device of aspect 1, wherein the luminescent emitters are covalently attached to at least a portion of the crystalline framework material.

Aspect 8. The device of aspect 1, wherein the crystalline framework material is a metal-organic framework comprising Cu₂(TCPP), where TCPP comprises 5,10,15,20-tetrakis(4-carboxyphenyl)porphyrin].

Aspect 9. A film comprising:
a) a crystalline framework material comprising a plurality of units cells having a crystal structure, wherein the crystalline framework material exhibits a preferred crystallographic orientation with respect to a planar surface of the film so that a majority of the unit cells are substantially uniformly oriented with respect to the planar surface; and
b) a plurality of luminescent emitters positioned in the crystalline framework material, wherein the luminescent emitters exhibit a non-random orientation with respect to the crystal structure of the unit cells, and the transition dipole moments of the luminescent emitters are oriented with respect to the planar surface of the film such that their orientation anisotropy factor, e, is less than 0.33.

Aspect 10. The film of aspect 9, wherein the crystalline framework material is a metal-organic framework, a covalent organic framework, or a porous coordination framework.

Aspect 11. The film of aspect 9, wherein the luminescent emitters are luminescent moieties selected from the group consisting of rare earth metal complexes, phenyl-based phosphines, phosphine oxides, substituted phosphines containing hydrogen, alkyls, alkenyls, alkynyls, aryls, amines, alkoxides, aryloxides, heterocyclic oxides, acyls, alkoxycarbonyls, aryloxycarbonyls, acyloxides, acylamines, alkoxycarbonylamines, aryloxycarbonylamines, sulfonylamines, sulfamoyls, carbamoyls, alkylthiols, arylthiols, heterocyclic thiols, and combinations thereof.

Aspect 12. The film of aspect 9, wherein the luminescent emitters are luminescent moieties selected from the group consisting of tetraphenylporphyrin platinum(II), (bppo)₂Ir(acac), (bppo)₂Ir(ppy), (ppy)₂Ir(bppo), (ppy)Re(CO)₃, (MDQ)₂Ir(acac), N,N′-di(1-naphthyl)-N,N′-diphenyl-(1,1′-biphenyl)-4,4′-diamine (NPD), and combinations thereof.

Aspect 13. The film of aspect 9, wherein at least 50% of the unit cells hold at least a respective one of the luminescent emitters substantially parallel to the planar surface of the film.

Aspect 14. The film of aspect 9, wherein at least 66% of the transition dipole moments are oriented substantially parallel to the planar surface with a maximum deviation of about +/−45°.

Aspect 15. The film of aspect 9, wherein the film is composed of one or more crystals comprising the unit cells of the crystalline framework material, each of the one or more crystals has at least one crystallographic axis that is aligned substantially parallel to a planar surface of the film, and the luminescent emitters are arranged in at least some of the unit cells such that the transition dipole moments of the luminescent emitters are substantially parallel to the crystallographic axis.

Aspect 16. The film of aspect 9, wherein the crystalline framework material comprises at least one metal-organic framework including metallic structural units linked by organic structural units, and wherein the luminescent emitters are comprised in at least a part of the organic structural units.

Aspect 17. The film of aspect 9, wherein the luminescent emitters reside in pores of the framework material.

Aspect 18. The film of aspect 9, wherein the luminescent emitters are covalently attached to at least a portion of the crystalline framework material.

Aspect 19. The film of aspect 9, wherein the crystalline framework material comprises a metal-organic framework comprising Cu₂(TCPP) where TCPP is 5,10,15,20-tetrakis(4-carboxyphenyl)porphyrin].

Aspect 20. A light-emitting device comprising an emission layer, and first and second electrodes between which a voltage can be applied to generate an electric field in at least part of the emission layer, wherein the emission layer comprises:
a) at least one crystal comprising a plurality of units cells of a crystalline framework material, wherein the crystal has at least one crystallographic axis that is oriented substantially parallel to a planar surface of the emission layer through which light is emitted; and
b) a plurality of luminescent emitters incorporated into at least some of the unit cells such that the transition dipole moments of the luminescent emitters are substantially parallel to the crystallographic axis.

Aspect 21. The device of aspect 20, wherein at least 50% of the unit cells hold at least one of the luminescent emitters substantially parallel to the crystallographic axis.

Aspect 22. The device of aspect 20, wherein at least 66% of the luminescent emitters have transition dipole moments that are oriented substantially parallel to the planar surface with a maximum deviation of +/−45°.

Aspect 23. The device of aspect 20, wherein the emission layer comprises a film composed of a plurality of crystals, and each crystal has at least one crystallographic axis that is oriented substantially parallel to the planar surface.

Aspect 24. The device of aspect 20, wherein the crystalline framework material comprises a metal-organic framework, and the luminescent emitters comprise luminophores modified by the addition of at least one coordinating group that allows each of the luminophores to act as a structural organic component in a MOF unit cell.

Aspect 25. The device of aspect 20, wherein the luminescent emitters reside in pores of the crystalline framework material, and the luminescent emitters are modified by addition of chemical moieties such as alkyl, aromatic, or halide groups.

Aspect 26. The device of aspect 20, wherein the luminescent emitters comprise luminophores modified by the addition of at least one functional group to allow the luminophores to bind to the unit cells through covalent or coordination bonds.

Aspect 27. The device of aspect 20, wherein the transition dipole moments are oriented substantially parallel to the planar surface so that their orientation anisotropy factor, Θ, is less than ⅓.

Aspect 28. The device of aspect 20, wherein the crystalline framework material is a metal-organic framework comprising Cu₂(TCPP), where TCPP comprises 5,10,15,20-tetrakis(4-carboxyphenyl)porphyrin].

Aspect 29. The device of aspect 20, wherein the crystalline framework material is a metal-organic framework, a covalent organic framework, or a porous coordination framework.

Aspect 30. A film comprising:
a) at least one crystal comprising a plurality of units cells of a crystalline framework material, wherein the crystal has at least one crystallographic axis that is oriented substantially parallel to a planar surface of the film; and
b) a plurality of luminescent emitters configured in at least some of the unit cells such that the transition dipole moments of the luminescent emitters are maintained in a substantially parallel orientation with respect to the crystallographic axis. In some embodiments, each of the unit cells that is functionalized with at least one of the luminescent emitters holds or positions the luminescent emitter in a substantially fixed orientation so that its transition dipole moment is oriented substantially parallel to the crystallographic axis.

Aspect 31. The film of aspect 30, wherein at least 30% of the unit cells hold at least one of the luminescent emitters such that its transition dipole has an orientation that is substantially parallel to the crystallographic axis. In some cases, at least 50%, 66%, 70%, 80% or 90% of the unit cells hold at least one of the luminescent emitters such that its transition dipole has an orientation that is substantially parallel to the crystallographic axis.

Aspect 32. The film of aspect 30, wherein at least 66% of the luminescent emitters have transition dipole moments that are oriented substantially parallel to the planar surface with a maximum deviation of about +/−45°.

Aspect 33. The film of aspect 30, wherein the film is composed of a plurality of crystals having the same crystallographic axis that is oriented substantially parallel to the planar surface.

Aspect 34. The film of aspect 30, wherein the transition dipole moments of the luminescent emitters are oriented substantially parallel to the planar surface of the film so that their orientation anisotropy factor, Θ, is less than ⅓.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g.; nanometers (nm), milligram (mg), milliliter (ml), millimeters (mm), p1, picoliter(s); s or sec, second(s); min, minute(s); h or hr, hour(s); and the like.

### Example 1-Materials and Methods for Fabricating Cu₂(TCPP) Film

Copper nitrate trihydrate (7 mg) and 5,10,15,20-tetrakis(4-carboxyphenyl)porphyrin] (TCPP, 30 mg) are dissolved in a solution of 1.5 mL diethylformamide and 0.5 mL ethanol. The resulting solution is sealed in a glass vial and placed in an oven. The oven is heated to 80° C. over a period of 30 minutes and held at 80° C. for a period of 12 hours. The oven is then cooled at a rate of 5° C./hour to room temperature. The resulting solution is centrifuged. The liquid contents are discarded and the red-brown solid, Cu₂(TCPP), is redispersed in 2 mL methanol. The centrifugation process is repeated two times. The resulting solution is diluted in methanol to a desired concentration, which may be one tenth the starting concentration, and deposited as a thin film onto a substrate. Thin film deposition may proceed as follows: The dispersion of Cu₂(TCPP) in methanol is pipetted dropwise onto the surface of water in a beaker. The MOF is observed to form a thin layer on top of the water. A glass slide is lowered onto the surface of the liquid such that the thin layer of MOF is deposited onto the surface of the glass. The glass slide is then submerged and removed from the water at an angle.

The description above illustrates embodiments of the disclosure by way of example and not necessarily by way of limitation. Many other embodiments and examples of the film, light-emitting devices, and methods for fabrication are possible. For example, suitable embodiments include, but are not limited to, any configuration of an OLED device in which the emission layer contains a crystalline framework material (e.g., a metal-organic framework) that is used to direct the orientation of luminescent moieties (e.g. phosphor molecules). This includes configurations such as presented in FIG. 5, as well as alternative configurations lacking any of the layers apart from the emission layer. This also includes configurations in which some of the layers are combined with the emission layer (e.g. a combined hole transport and emission layer). This also includes configurations with just two electrodes and an emission layer as in FIG. 2.

Accordingly, the scope of the present disclosure should be determined by the following claims and their legal equivalents. Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A lighting device comprising:
a) an emission layer comprising a luminescent film; and
b) first and second electrodes between which a voltage can be applied to generate an electric field in at least part of the emission layer, wherein the film is composed of at least one crystal comprising repeating structural units of a crystalline framework material, the crystal has at least one crystallographic axis that is aligned substantially parallel to a planar surface of the luminescent film with a maximum deviation of up to +/−35° from this parallel orientation, and luminescent emitters having transition dipole moments are arranged in at least 30% of the repeating structural units of the crystalline framework material such that the transition dipole moments of the luminescent emitters are configured in a substantially parallel orientation relative to the crystallographic axis with a maximum deviation of up to +/−350 from this parallel orientation.

2. The device of claim 1, wherein the luminescent emitters are configured to emit light from the planar surface of the film at an average angle of emission of 450 or less from a surface normal to the planar surface.

3. The device of claim 1, wherein the luminescent emitters are configured to be substantially uniformly oriented relative to the planar surface such that their transition dipole moments have an orientation anisotropy factor (0) of less than 0.33.

4. The device of claim 1, wherein at least 50% of the repeating structural units comprise a luminescent emitter having a transition dipole moment configured to be substantially parallel to the planar surface of the film.

5. The device of claim 1, wherein at least 66% of the transition dipole moments of the luminescent emitters are oriented substantially parallel to the planar surface of the film with a maximum deviation of about +/−45°.

6. The device of claim 1, further comprising a controller or voltage source for applying the voltage (potential difference) between the first and second electrodes.

7. The device of claim 6, wherein the controller is arranged to apply an AC voltage having a waveform that is a square wave, a triangle wave, or a pulsed wave.

8. The device of claim 1, wherein the crystalline framework material comprises at least one metal-organic framework, covalent organic framework, porous coordination framework or porous coordination polymer.
